(19) United States
(12) Patent Application Publication
(10) Pub. No.: US 2022/0325229 A1
(43) Pub. Date: Oct. 13, 2022

(54) MICRONUTRIENT-CONTAINING BIOSTIMULANT COMPOSITION

(71) Applicant: Shared-X LLC, Miami, FL (US)

(72) Inventor: Jose Antonio de Cote, Miami, FL (US)

(21) Appl. No.: 17/658,643

(22) Filed: Apr. 8, 2022

Related U.S. Application Data
(60) Provisional application No. 63/201,060, filed on Apr. 9, 2021.

(51) Int. Cl. C12M 1/00; C05D 9/02; C05G 5/23; C05B 17/02

(52) U.S. Cl. CPC C12M 45/06; C05D 9/02; C05G 5/23; C05B 17/02

(57) ABSTRACT

A biostimulant composition including selected amino acids and oligopeptides derived from legumes by enzymatic hydrolysis and added micronutrients is provided.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in their entireties and for all purposes.

### BACKGROUND

During agricultural crop generation, plants grow under conditions that may be particularly efficient and/or productive. However, some crop plants grow under conditions that present nutrient deficiencies that hinder or prevent healthy, efficient growth. Biostimulants may be used to address nutrient deficiencies of certain plants.

The background description provided herein is for the purposes of generally presenting the context of the disclosure. Work of the presently named inventors, to the extent it is described in this background section, as well as aspects of the description that may not otherwise constitute prior art at the time of filing, are neither expressly nor impliedly admitted as prior art against the present disclosure.

### SUMMARY

One aspect involves a biostimulant composition for applying to plants, the biostimulant composition including: two or more free amino acids derived from a plant by enzymatic hydrolysis, the two or more free amino acids being one or more of glutamic acid, glycine, and lysine; oligopeptides derived from the plant by enzymatic hydrolysis; and one or more nutrients comprising at least one micronutrient being one or more of iron, manganese, boron, molybdenum, zinc, chlorine, sodium, and cobalt, whereby the plant is a non-carob plant.

In various embodiments, the one or more nutrients includes a macronutrient such as one or more of potassium and calcium. In some embodiments, the biostimulant composition also includes water, such that the macronutrient has a concentration of at least about 5%. In some embodiments, the biostimulant composition also includes water, such that the macronutrient is potassium and has a concentration of at least about 10%.

In various embodiments, the at least one micronutrient includes boron. In some embodiments, the biostimulant composition also includes water, such that the boron has a concentration of at least about 5%.

In various embodiments, the at least one micronutrient includes manganese. In some embodiments, the biostimulant composition also includes water, such that the manganese has a concentration of at least about 4%.

In various embodiments, the one or more nutrients includes magnesium. In some embodiments, the biostimulant composition also includes water, such that the magnesium has a concentration of at least about 4%.

In various embodiments, the at least one micronutrient includes zinc. In some embodiments, the biostimulant composition also includes water, such that the zinc has a concentration of at least about 6%.

In various embodiments, the at least one micronutrient includes zinc and manganese. In some embodiments, the biostimulant composition also includes water, such that the zinc has a concentration of at least about 3%, and the manganese has a concentration of at least about 3%.

In various embodiments, the plant is one or more of peanut, tarwi, carob germ, soybean, and Plukenetia volubilis.

In various embodiments, the biostimulant composition also includes water. In some embodiments, volume ratio of the water to the biostimulant composition is about 0.7 to about 1.3.

In various embodiments, wherein the two or more free amino acids include at least glutamic acid and glutamine, glycine, threonine, alanine, leucine, and lysine.

In various embodiments, the two or more free amino acids include trace amounts of one or more secondary amino acid such as one or more of aspartic acid, serine, tyrosine, arginine, valine, tryptophan, phenylalanine, asparagine, and isoleucine.

In various embodiments, the two or more free amino acids include less than 1% of histidine, methionine, glutamine, proline, hydroxyproline, ornithine, taurine, and combinations thereof.

In various embodiments, the biostimulant composition also includes one or more phytohormones derived from the plant. In some embodiments, the one or more phytohormones are one or more of cytokinins, abscisic acids (ABAs), jasmonates, auxins, and phenolics. In some embodiments, the one or more phytohormones include phenolics. In some embodiments, the one or more phytohormones included dihydrophaseic acid (DPA) and phenylacetic acid (PAA).

In various embodiments, the biostimulant composition has a density of about 1 gr/ml to about 1.3 gr/ml.

In various embodiments, the enzymatic hydrolysis is catalyzed by a protease.

In various embodiments, the at least one micronutrient has a concentration of at least about 3% by weight of element by volume of the biostimulant composition.

Another aspect involves a method of preparing the biostimulant composition of any of the preceding embodiments.

Another aspect involves a method of applying the biostimulant composition of any of the preceding embodiments to a plant, the method including delivering the biostimulant composition to plants via irrigation.

Another aspect involves a method of applying the biostimulant composition of any of the preceding embodiments to a plant, the method including delivering the biostimulant composition to plants via a mister.

Another aspect involves a biostimulant composition for applying to plants, the biostimulant composition including: free amino acids including: free glutamic acid and glutamine having a weight percent of about 30% to about 40% of the total free amino acid weight in the biostimulant composition, glycine having a weight percent of about 10% to about 20% of the total free amino acid weight in the biostimulant composition, and lysine having a weight percent of about 40% to about 60% of the total free amino acid weight in the biostimulant composition; at least one oligopeptide; and a nutrient.

In various embodiments, the nutrient is one or more of calcium, potassium, sulfur, magnesium, carbon, oxygen, hydrogen, iron, manganese, boron, molybdenum, zinc, chlorine, sodium, and cobalt.

In various embodiments, the biostimulant composition also includes a phytohormone. In some embodiments, the phytohormone is one or more of cytokinins, abscisic acids (ABAs), jasmonates, auxins, and phenolics.

Another aspect involves a method of producing a biostimulant composition including: receiving a plant-based feedstock including a plant protein; enzymatically hydrolyzing the plant protein to produce a hydrolysis product comprising an amino acid and an oligopeptide; and adding one or more nutrients to the plant-based feedstock or hydrolysis product.

In various embodiments, the plant-based feedstock is one or more of legumes, tarwi, peanut, and Plukenetia volubilis.

In various embodiments, the one or more nutrients are one or more of calcium, potassium, sulfur, magnesium, carbon, oxygen, hydrogen, iron, manganese, boron, molybdenum, zinc, chlorine, sodium, and cobalt.

In various embodiments, the amino acid is one or more isting of glutamic acid, glycine, threonine, alanine, leucine, lysine, and combinations thereof.

In various embodiments, the hydrolysis product also includes one or more phytohormones.

In various embodiments, the one or more phytohormones are one or more of cytokinins, abscisic acids (ABAs), jasmonates, auxins, and phenolics.

Another aspect involves a biostimulant composition for applying to plants, the biostimulant composition including: free amino acids derived from a plant by enzymatic hydrolysis; and one or more nutrients being one of calcium, potassium, iron, manganese, boron, molybdenum, zinc, chlorine, sodium, cobalt, silicon, magnesium, and combinations thereof.

These and other aspects are described further below with reference to the drawings.

### BRIEF DESCRIPTION OF THE DRAWINGS

FIG. 1 is a schematic illustration of components of a biostimulant composition in accordance with certain disclosed embodiments.

FIG. 2 is a process flow diagram depicting operations performed in a method performed in accordance with certain disclosed embodiments.

FIG. 3 is a process flow diagram depicting operations performed in a method performed in accordance with certain disclosed embodiments.

FIGS. 4A and 4B are schematic illustrations depicting example techniques for applying a biostimulant composition in accordance with certain disclosed embodiments.

FIG. 5 is a schematic illustration of an enzymatic hydrolysis reactor that may be used to perform certain disclosed embodiments.

### DETAILED DESCRIPTION

In the following description, numerous specific details are set forth to provide a thorough understanding of the presented embodiments. The disclosed embodiments may be practiced without some or all of these specific details. In other instances, well-known process operations have not been described in detail to not unnecessarily obscure the disclosed embodiments. While the disclosed embodiments will be described in conjunction with the specific embodiments, it will be understood that it is not intended to limit the disclosed embodiments.

Agricultural crop generation involves consideration of various factors to ensure healthy and productive growth of the crops, including the geographical location and growth conditions. However, crops may encounter various agricultural growth difficulties, including soil contamination, genetic mutations, pests (such as insects), disease (e.g., fungal, bacterial, and viral diseases), disruptive effects of automated techniques (e.g., tilling, planting, harvesting, watering, etc.), and other non-ideal growing conditions such as soil composition, humidity (excessive or very low), temperature (very high or very low), luminosity level (e.g., excess solar luminosity or lack thereof), flooding and/or drought, stress caused by fertilizers, inadequate pollination, excess of soil salts (e.g., minerals), and lack of organic material and/or minerals in the soil.

To help resolve these agricultural growth difficulties, it is useful to use substances that are compatible with the plants. One type of compatibility that may be used considers the types of amino acids that the plant generates to sustain life. During the agricultural growth process, plants spend energy manufacturing certain amino acids that are important for their well-being. Biostimulants and/or nutritional correctors can supply these amino acids and allow the plant to redirect its energy to performing other functions. Application of biostimulants may reduce negative impacts of biotic stressors as well as abiotic stressors and help correct micronutrient and/or macronutrient deficiencies in the plant. Biostimulant compositions described herein have an amino acid profile. That profile may be based, at least in part, on an initial feedstock used to make the biostimulant composition. Example feedstocks include plant waste (e.g., husks or seedpods) and plants having limited economic value.

It has been observed that some biostimulant compositions, such as those derived from rice, do not have an amino acid profile that matches the needs of some plants growing under some conditions. Additionally, some biostimulant compositions are created using acid hydrolysis, which often destroys certain nutrients and/or amino acids in the feedstock, which can generate free amino acids that may be useful to a plant. While animal derived biostimulants may be generated, such biostimulant compositions lack some components such as phytohormones that are beneficial for plant growth. Further, it is generally more difficult to break down proteins from animal feedstock than from plant feedstock. Some biostimulant compositions may also not be suitable because of synthesis difficulties, lack of efficiency in generating the composition, cost of production, and environmental condition limitations.

Provided herein are biostimulant compositions that contain multiple amino acids and one or more nutrients such as micronutrients and macronutrients. Biostimulant compositions disclosed herein may include one or more components that act as a secondary metabolite. Certain disclosed biostimulant compositions include oligopeptides that may bioencapsulate micronutrients and/or macronutrients. In certain embodiments, biostimulant compositions are produced from feedstocks that generate an amino acid profile suitable for plants of many types.

Some feedstocks that may be used have organic origin that have traditionally been considered directly as waste, or at most, are considered low added value materials. These different agro-industrial by-products have properties that give them great potential for application in the agricultural biotechnology industry.

These starting materials are not easily usable, as they are not accessible or available. For example, its high insolubility, mainly, makes its use difficult. However, enzyme technology, with extraction and/or modification processes, can convert these organic materials into new products with greater functionality, due to the concentration of active principles, and better application technological properties (increased solubility and decreased molecular size of its components).

Amino acids generated from feedstock may include free amino acids, amino acids in forming peptides, and amino acids in a protein. Free amino acids are derived from protein hydrolysis and are not bound to any other amino acids through peptide bonds. Due to the low molecular weight of free amino acids, plants are able to assimilate free amino acids quickly and their effects on plant metabolism are more defined. Therefore, free amino acids can be important in plant nutrition. Of note, when two or more amino acids are joined together (by a peptic bond), they form a peptide. The longer the length of the peptide (more amino acids attached), the more difficult will be the direct assimilation by plants. Lastly, amino acids may be present in a protein. The union of the different polypeptide chains forms a protein. The structural units of proteins are the amino acids joined in a sequence and the characteristic order for each type of protein. Free amino acids and some low molecular weight peptides are useful as products applied to plants. The percentage of each type of amino acids depends on the type of hydrolysate and the origin of the proteins (animal or vegetable), and with it, the quality of the final product.

In certain embodiments, the feedstock contains plant material such material from a carob plant, a peanut plant, a lupin plant, a soybean plant, a rice plant, or the like. Sources that have a high concentration of vegetable protein can be used in various embodiments. When biostimulant compositions are produced from plant feedstock, acid hydrolysis is not used. Some disclosed biostimulant compositions are produced by enzymatic hydrolysis of plant feedstock.

### Definitions

Unless defined otherwise, all technical and scientific terms used herein have the same meaning as commonly understood by one of ordinary skill in the art. The terms presented immediately below are more fully understood by reference to the remainder of the specification. The following descriptions are presented to facilitate understanding of certain embodiments and the complex concepts described herein. These descriptions are not intended to limit the full scope of the disclosure.

“Biostimulant composition” or “nutritional corrector composition” may refer to a composition, which may be a substance or mixture, that supplements or corrects nutritional deficiencies in a plant to improve the function of the plant by stimulating biological processes, improving the availability of nutrients, optimizing the plants' absorption of nutrients, increase tolerance to abiotic stresses, and/or improve quality aspects of the harvest.

“Micronutrient” may refer to a secondary plant nutrient used in smaller amounts for nourishment and growth of a plant. A plant nutrient is secondary if a plant only uses trace amounts of it to sustain life. Examples of micronutrients include iron, manganese, zinc, copper, boron, and molybdenum.

“Macronutrient” may refer to a plant nutrient used in large amounts for nourishment and growth of a plant. Examples of primary macronutrients are nitrogen, phosphorous, and potassium. Examples of secondary macronutrients are magnesium, sulfur, and calcium.

A “peptide” may refer to a linear chain of amino acids linked by amide-type chemical bonds, which are called peptide bonds. Thus, to form peptides, amino acids are linked together forming chains of variable length and sequence. Dipeptides may refer to a linear chain of two amino acids linked by a peptide bond. Tripeptides may refer to a linear chain of three amino acids, and tetrapeptides may refer to a linear chain of four amino acids.

An “oligopeptide” may refer to a peptide having less than 10 amino acids.

“Amino acid profile” may refer to the amounts of the amino acids present in a composition. Amino acid profiles may be qualitative or quantitative. Qualitative amino acid profiles identify which amino acids are present in a composition. Quantitative amino acid profiles refer to the relative amounts of amino acids present in a composition and/or to the absolute amounts of amino acids present in a composition.

“Free amino acid” or “free amino acid component” may refer to an amino acid that is not bound to other amino acids and/or peptides via peptide bonds.

A “primary amino acid component” may refer to an amino acid in a composition that is at least about 1% (w/w) of the total weight of amino acids in a composition. In some embodiments, a primary amino acid component is at least about 10% (w/w) of the total weight of amino acids in a composition.

A “secondary amino acid component” may refer to an amino acid in a composition that has a concentration of less than about 1% (w/w) of the total weight of amino acids in a composition. In some embodiments, a secondary amino acid component is greater than about 0.01% and less than 0.7% (w/w) of total weight of amino acids in a composition.

“Feedstock” may refer to a raw, unprocessed material source that can be processed and/or broken down to generate nutritional components.

“Enzymatic hydrolysis” may refer to a process which enzymes are used to facilitate degradation of a feedstock by hydrolytically cleaving bonds in molecules with the addition of the elements of water. Proteases are sometimes used to perform enzymatic hydrolysis on a protein-containing feedstock.

Biostimulant Compositions

In certain embodiments, a biostimulant composition includes two or more amino acids and one or more micronutrients. Biostimulant compositions have amino acid profiles and oligopeptide profiles. Biostimulant compositions in accordance with certain disclosed embodiments are derived from feedstock that includes a plant-based protein source. Plant-based protein sources may be selected based on their high organic matter content. These by-products have been selected by virtue of their high organic matter content, mainly proteins, and have been characterized to carry out enzymatic hydrolysis processes, obtaining said biostimulant products.

Through hydrolytic processes, the functional properties of organic matter contained in agro-industrial organic by-products has been modified, which provides them with a greater capacity for agricultural application, by increasing their bioavailability.

Either or both of the amino acids and oligopeptides may originate from a plant-based protein source. Some biostimulants contain other components from a plant source such as secondary metabolites, phytohormones, micronutrients, and/or macronutrients. Certain biostimulant compositions described herein are in liquid form or have components that are suspended in liquids. Certain biostimulant compositions described herein are in solid form or have solid components.

Some plant-based protein sources include but are not limited to plant material from the Fabaceae and/or Leguminosae family. Particular examples of plant-based protein sources include plant material from the Ceratonia genus, the Arachis genus, the Lupinus genus, the Glycine genus and the Pisum genus. For example, carob germ or carobs (Ceratonia siliqua) may be a suitable plant-based protein source. Peanuts (Arachis hypogaea) may also be a suitable plant-based protein source. Tarwi (Lupinus mutabilis) may also be a suitable plant-based protein source. Soybean (Glycine max) may also be a suitable plant-based protein source. Peas (Pisum sativum) may also be a suitable plant-based protein source. Other suitable genera that may provide a protein source include but are not limited to Astragalus, Acacia, Indigofera, Crotalaria, and Mimosa.

Some plant-based protein sources may be from the Euphorbiaceae family. An example genus from this family is the Plukenetia genus. Plukenetia volubilis, or Sacha inchi, is a perennial plant that is native to tropical South America. Plukenetia volubilis may also be a suitable plant-based protein source as it may have significant protein content as well as omega-3 fatty acids, omega-6 fatty acids, and omega-9 fatty acids.

Some plant-based protein sources may be from the Poaceae family. One example genus from this family is the Oryza genus. For example, rice may be a suitable plant-based protein source.

In various embodiments, parts of a plant may be used as the plant-based protein source. Example sources include but are not limited to roots, stems, husks, leaves, and seeds. In certain embodiments, plant feedstock is used with little or no preparation other than harvesting and optionally storing and/or milling. In some embodiments, plant feedstock is subject to a post-harvest process such as high temperature drying, oil extraction, or similar process. In peanut sources, after oil extraction, the remaining dry “cake” is used as feedstock. In carob sources, the whole seed with the husk is dried and milled to form the feedstock. In lupine sources, the beans are dried and milled to form the feedstock.

In some embodiments, the plant-based protein source may have at least about 60% protein content by weight of the prepared feedstock (such as dry cake of peanut feedstock), or at least about 50% protein content by weight, or at least about 30% protein content by weight.

Biostimulant compositions have an amino acid profile. The amino acid profile is different depending on the starting raw material and the hydrolysis conditions. Additionally, some raw materials will generate different peptide profiles, and some peptide profiles (oligopeptides and/or polypeptides) have greater or lesser beneficial properties such as nutrient, antimicrobial, and antibacterial capacity. When controlled enzymatic hydrolysis of proteins is carried out, a balance is obtained between amino acids in free form and in peptides, which gives the hydrolysate a significant nutritional role as a biostimulant, due to its ability to stimulate the growth and development of plants and crops, as well as increase and enhance the microbiological activity of the soil. The amino acids and the low molecular weight peptides that make them up are nutritious substances that are easily absorbed and assimilated by plants, both by foliar and root routes, and can be transported to the plant's organs, such as buds, flowers, fruits.

Various types of amino acids may be present in the biostimulant composition. An amino acid profile may be characterized by relative amounts or concentrations of individual amino acids (e.g., proline, alanine, arginine) and/or by the relative amounts or concentrations of classes or types of amino acids.

In some embodiments, amino acids may be non-proteinogenic amino acids. In some embodiments, amino acids may be proteinogenic amino acids. For example, in some embodiments, any one or more of the following types of amino acids are present: aliphatic amino acids, aromatic amino acids, non-polar and neutral amino acids, polar and neutral amino acids, acidic and polar amino acids, and basic and polar amino acids. The amino acids in a biostimulant composition may be proteinogenic or non-proteinogenic (e.g., taurine and ornithine). A biostimulant composition may have at least one of the following amino acids at greater than a trace concentration: aspartic acid with asparagine, glutamic acid with glutamine, glycine, serine, threonine, histidine, tyrosine, arginine, alanine, methionine, valine, tryptophan, phenylalanine, asparagine, glutamine, isoleucine, leucine, proline, hydroxyproline, ornithine, and taurine. In some embodiments, the biostimulant composition includes at least glycine and lysine. In some embodiments, the biostimulant includes at least glutamic acid, glutamine, glycine, and lysine.

The below concentration percentages are percentages by weight of each amino acid divided by the total weight of the free amino acid component in the biostimulant composition (e.g., 33% glutamic acid and glutamine means that of the weight of amino acids in the biostimulant composition, 33% of the weight is glutamic acid and glutamine) Ranges in Table 1 are approximate concentration ranges for each amino acid produced from one example raw feedstock material.

TABLE 1
Amino Acid Profile

| Amino Acid | Minimum | Maximum |
|---|---|---|
| Aspartic acid and asparagine | About 0.05% | About 0.3% |
| Glutamic acid and glutamine | About 30% | About 40% |
| Glycine | About 10% | About 20% |
| Serine | About 0.1% | About 0.5% |
| Threonine | About 0.3% | About 0.7% |
| Histidine | About 0.01% | About 0.1% |
| Tyrosine | About 0.01% | About 0.2% |
| Arginine | About 0.1% | About 0.5% |
| Alanine | About 0.3% | About 0.7% |
| Methionine | About 0.01% | About 0.1% |
| Valine | About 0.1% | About 0.5% |
| Tryptophan | About 0.1% | About 0.5% |
| Phenylalanine | About 0.1% | About 0.5% |
| Asparagine | About 0.1% | About 0.5% |
| Glutamine | About 0.01% | About 0.1% |
| Isoleucine | About 0.1% | About 0.5% |
| Leucine | About 0.3% | About 0.7% |
| Lysine | About 40% | About 60% |
| Proline | About 0.01% | About 0.1% |
| Hydroxyproline | About 0.01% | About 0.1% |
| Omithine | About 0.01% | About 0.1% |
| Taurine | About 0.01% | About 0.1% |

In various embodiments, glutamine, histidine, hydroxyproline, methionine, omithine, proline, taurine, tyrosine, aspartic acid and asparagine, arginine, asparagine, phenylalanine, serine, tryptophan, valine, isoleucine, alanine, leucine, and threonine may be secondary amino acid components. In various embodiments, lysine, glycine, and glutamic acid and glutamine may be primary amino acid components.

In some embodiments, the free amino acid component of a biostimulant composition includes (a) one or more primary amino acid components selected from the group consisting of lysine, glycine, and glutamic acid and glutamine, and (b) one or more secondary amino acid components selected from the group consisting of glutamine, histidine, hydroxyproline, methionine, omithine, proline, taurine, tyrosine, aspartic acid and asparagine, arginine, asparagine, phenylalanine, serine, tryptophan, valine, isoleucine, alanine, leucine, and threonine. In some embodiments, the free amino acid component of a biostimulant composition includes (a) one or more primary amino acid components selected from the group consisting of lysine, glycine, and glutamic acid and glutamine, and (b) one or more secondary amino acid components selected from the group consisting of alanine, leucine, and threonine. In some embodiments, the free amino acid component of a biostimulant composition includes (a) one or more primary amino acid components selected from the group consisting of lysine, glycine, and glutamic acid and glutamine, and (b) one or more secondary amino acid components selected from the group consisting of tyrosine, aspartic acid and asparagine, arginine, asparagine, phenylalanine, serine, tryptophan, valine, and isoleucine.

In various embodiments, the free amino acid component of a biostimulant composition has less than about 0.1% histidine, less than about 0.1% methionine, less than about 0.1% glutamine, less than about 0.1% proline, less than about 0.1% hydroxyproline, less than about 0.1% omithine, less than about 0.1% taurine by weight, or any combination of these. In some embodiments, the free amino acid component of a biostimulant composition has less than about 0.1% histidine by weight, or about 0.01% to about 0.1% histidine by weight of the total weight of free amino acid components in the biostimulant composition. In some embodiments, the free amino acid component of a biostimulant composition has less than about 0.1% methionine by weight of the total weight of free amino acid components in the biostimulant composition, or about 0.01% to about 0.1% methionine by weight of the total weight of free amino acid components in the biostimulant composition. In some embodiments, the free amino acid component of a biostimulant composition has less than about 0.1% glutamine by weight of the total weight of free amino acid components in the biostimulant composition, or about 0.01% to about 0.1% glutamine by weight of the total weight of free amino acid components in the biostimulant composition. In some embodiments, the free amino acid component of a biostimulant composition has less than about 0.1% proline by weight of the total weight of free amino acid components in the biostimulant composition, or about 0.01% to about 0.1% proline by weight of the total weight of free amino acid components in the biostimulant composition. In some embodiments, the free amino acid component of a biostimulant composition has less than about 0.1% hydroxyproline by weight of the total weight of free amino acid components in the biostimulant composition, or about 0.01% to about 0.1% hydroxyproline by weight of the total weight of free amino acid components in the biostimulant composition. In some embodiments, the free amino acid component of a biostimulant composition has less than about 0.1% omithine by weight of the total weight of free amino acid components in the biostimulant composition, or about 0.01% to about 0.1% omithine by weight of the total weight of free amino acid components in the biostimulant composition of the total weight of free amino acid components in the biostimulant composition. In some embodiments, the free amino acid component of a biostimulant composition has less than about 0.1% taurine by weight of the total weight of free amino acid components in the biostimulant composition, or about 0.01% to about 0.1% taurine by weight of the total weight of free amino acid components in the biostimulant composition.

In some embodiments, about 0.01% to about 0.3% of the free amino acid components in the biostimulant composition is aspartic acid and asparagine by weight. In some embodiments, the free amino acid component of a biostimulant composition has about 0.01% to about 0.2% tyrosine by weight.

In various embodiments, the free amino acid component of a biostimulant composition has about 0.1% to about 0.5% of each of serine, arginine, isoleucine, valine, tryptophan, phenylalanine, and asparagine by weight. In some embodiments, the free amino acid component of a biostimulant composition has about 0.1% to about 0.5% serine by weight of the total weight of free amino acid components in the biostimulant composition. In some embodiments, the free amino acid component of a biostimulant composition has about 0.1% to about 0.5% arginine by weight of the total weight of free amino acid components in the biostimulant composition. In some embodiments, the free amino acid component of a biostimulant composition has about 0.1% to about 0.5% valine by weight of the total weight of free amino acid components in the biostimulant composition. In some embodiments, the free amino acid component of a biostimulant composition has about 0.1% to about 0.5% tryptophan by weight of the total weight of free amino acid components in the biostimulant composition. In some embodiments, free amino acid component of a the biostimulant composition has about 0.1% to about 0.5% phenylalanine by weight of the total weight of free amino acid components in the biostimulant composition. In some embodiments, the free amino acid component of a biostimulant composition has about 0.1% to about 0.5% asparagine by weight of the total weight of free amino acid components in the biostimulant composition. In some embodiments, the free amino acid component of a biostimulant composition has about 0.1% to about 0.5% isoleucine by weight of the total weight of free amino acid components in the biostimulant composition.

In various embodiments, the free amino acid component of a biostimulant composition has about 0.3% to about 0.7% of each of threonine, alanine, and leucine by weight. In some embodiments, the free amino acid component of a biostimulant composition includes about 0.3% to about 0.7% threonine by weight of the total weight of free amino acid components in the biostimulant composition. In some embodiments, the free amino acid component of a biostimulant composition includes about 0.3% to about 0.7% alanine by weight of the total weight of free amino acid components in the biostimulant composition. In some embodiments, the free amino acid component of a biostimulant composition includes about 0.3% to about 0.7% leucine by weight of the total weight of free amino acid components in the biostimulant composition.

In various embodiments, the free amino acid component of a biostimulant composition has mostly lysine, or about 50% or more lysine, by weight of the total weight of free amino acid components in the biostimulant composition. In various embodiments, the free amino acid component of a biostimulant composition includes mostly glycine, glutamic acid and glutamine, and lysine. In some embodiments, about 30% to about 40% of the free amino acid component of a biostimulant composition is glutamic acid and glutamine. In some embodiments, about 10% to about 20% of the free amino acid component of a biostimulant composition is glycine. In some embodiments, about 40% to about 60% of the free amino acid component of a biostimulant composition is lysine.

The biostimulant composition may include alpha amino acids. The biostimulant composition may include L-alpha amino acids. The biostimulant composition may include basic amino acids. The biostimulant composition may include aliphatic amino acids. The biostimulant composition may include charge-neutral polar amino acids.

The biostimulant composition may include one or more oligopeptides. An oligopeptide may facilitate delivering nutrients to plants and/or moving nutrients within plants.

The biostimulant composition may optionally include one or more non-amino acid and non-peptide components from the plant-based protein source. Examples of such additional plant-based components include phytohormones and secondary metabolites. Example phytohormones include cytokinins, abscisic acid, jasmonates, auxins, and phenolics. Example cytokinins include but are not limited to trans-zeatin riboside (tZR), dihydrozeatin riboside (DZR), cis-zeatin (cZ), cis-zeatin riboside (cZR), isopentenyl adenine (iP), isopentenyl adenosine (iPR), 2-methylthio zeatin (MeS-Z), and 2-methylthio isopentenyl adenine (MeS-iP). Example abscisic acids include abscisic acid (ABA), phaseic acid (PA), dihydrophaseic acid (DPA), and 9-hydroxy-ABA (90H-ABA). Example jasmonates include jasmonic acid (JA) and jasmonic acid isoleucine (JA-Ile). Example auxins include indole-3-acetic acid (IAA), oxo-indole-3-acetic acid (Ox1AA), and indole-3-acetamide (IAM). Example phenolics include salicylic acid (SA) and phenylacetic acid (PAA).

In some embodiments, the concentration of one or more cytokinins in the composition is about 0.5 pmol/ml to about 15 pmol/ml. In some embodiments, the concentration of tZR is about 0.1 pmol/ml to about 0.4 pmol/ml. In some embodiments, the concentration of DZR is about 0.5 pmol/ml to about 1.2 pmol/ml. In some embodiments, the concentration of cZ is about 6 pmol/ml to about 8 pmol/ml. In some embodiments, the concentration of cZR is about 1 pmol/ml to about 2 pmol/ml. In some embodiments, the concentration of iP is about 10 pmol/ml to about 15 pmol/ml. In some embodiments, the concentration of iPR is about 1 pmol/ml to about 2 pmol/ml. In some embodiments, the concentration of MeS-Z is about 4 pmol/ml to about 6 pmol/ml. In some embodiments, the concentration of MeS-iP is about 0.5 pmol/ml to about 0.1 pmol/ml.

In one example, the concentration of tZR is about 0.2 pmol/ml. In one example, the concentration of DZR is about 1 pmol/ml. In one example, the concentration of cZ is about 8 pmol/ml. In one example, the concentration of cZR is about 2 pmol/ml. In one example, the concentration of iP is about 14 pmol/ml. In one example, the concentration of iPR is about 1 pmol/ml. In one example, the concentration of MeS-Z is about 5 pmol/ml. In one example, the concentration of MeS-iP is about 1 pmol/ml.

In some embodiments, the concentration of certain ABAs may range from about 0.1 pmol/ml to about 2800 pmol/ml. In some embodiments, the concentration of ABA is about 3 pmol/ml to about 5 pmol/ml. In some embodiments, the concentration of PA is about 0.1 pmol/ml to about 0.2 pmol/ml. In some embodiments, the concentration of DPA is about 2500 pmol/ml to about 2800 pmol/ml. In some embodiments, the concentration of 90H-ABA is about 0.5 pmol/ml to about 1.0 pmol/ml.

In some embodiments, the concentration of ABA is about 4 pmol/ml. In some embodiments, the concentration of PA is about 0.1 pmol/ml. In some embodiments, the concentration of DPA is about 2700 pmol/ml. In some embodiments, the concentration of 90H-ABA is about 0.7 pmol/ml.

In some embodiments, the concentration of certain jasmonates may range from about 0.1 pmol/ml to about 3 pmol/ml. In some embodiments, the concentration of JA is about 2 pmol/ml to about 3 pmol/ml. In some embodiments, the concentration of JA-Ile is about 0.1 pmol/ml to about 0.4 pmol/ml.

In one example, the amount of JA is about 3 pmol/ml. In one example, the amount of JA-Ile is about 0.3 pmol/ml.

In some embodiments, the content of certain auxins may range from about 3 pmol/ml and about 20 pmol/ml. In some embodiments, the amount of IAA is about 15 pmol/ml to about 20 pmol/ml. In some embodiments, the amount of OxIAA is about 4 pmol/ml to about 5 pmol/ml. In some embodiments, the amount of IAm is about 3 pmol/ml to about 5 pmol/ml.

In some embodiments, the amount of IAA is about 18 pmol/ml. In some embodiments, the amount of OxIAA is about 5 pmol/ml. In some embodiments, the amount of IAM is about 5 pmol/ml.

In some embodiments, the content of certain phenolics is about 150 pmol/ml to about 50000 pmol/ml. In some embodiments, phenolics are the majority phytohormone of all phytohormones in the biostimulant composition. In some embodiments, the amount of SA is about 150 pmol/ml to about 200 pmol/ml. In some embodiments, the amount of PAA is about 40000 pmol/ml to about 50000 pmol/ml.

In some embodiments, the amount of SA is about 182 pmol/ml. In some embodiments, the amount of PAA is about 46000 pmol/ml.

In some embodiments, the portion of the biostimulant composition having phytohormones may be predominantly abscisic acids and phenolics. In some embodiments, phenolics are the majority component of phytohormones in a biostimulant composition.

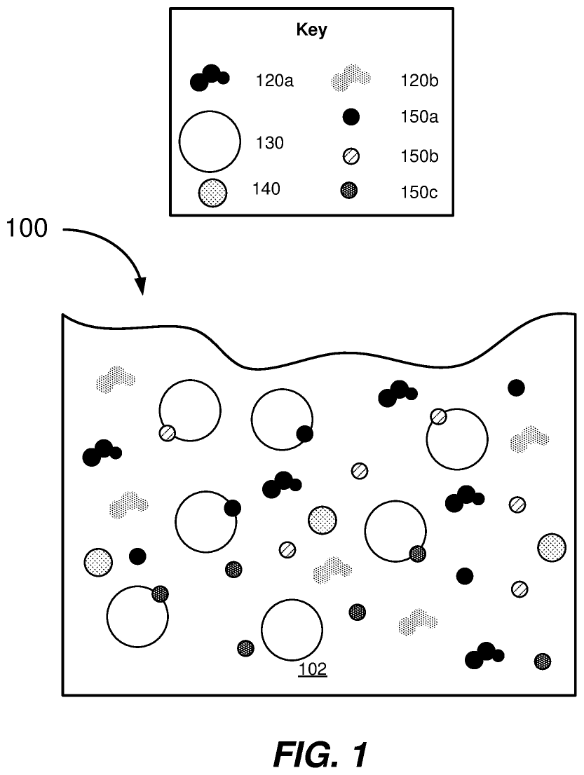

FIG. 1 shows an example schematic illustration of components of a biostimulant composition with components suspended in a liquid in accordance with certain disclosed embodiments. FIG. 1 includes composition 100 having a liquid 102 with suspended components. Suspended components include various types of free amino acids which are depicted as a first type of amino acid 120a and a second type of amino acid 120b. Although two types are depicted in this figure, it will be understood by a person of skill in the art that many types of free amino acids may be in the liquid 102 depending on the amino acid profile, and that the relative concentrations of the free amino acids may vary. Liquid 102 also includes micronutrients 150a, 150b, and 150c. Liquid 102 also includes macronutrient 140. Although three types of micronutrients and one type of macronutrient are depicted in this figure, it will be understood by a person of skill in the art that more or fewer types of micronutrients and more or fewer types of macronutrients may be presented in the composition 100. In various embodiments, manganese, boron, zinc, and mixtures of zinc and manganese may be one or more of micronutrients 150a, 150b, and 150c. In some embodiments, only one type of micronutrient (e.g., manganese, boron, or zinc) is present. In some embodiments, mixtures of micronutrients (e.g., zinc and manganese) are present. In various embodiments, calcium or potassium may be macronutrient 140. In some embodiment, only one type of macronutrient is added and no additional micronutrients 150a, 150b, or 150c are added, but some micronutrients from the original feedstock itself may be present. In some embodiments, some micronutrients 150a, 150b, and 150c and/or macronutrients 140 may be derived from the plant-based protein source. In some embodiments, some micronutrients 150a, 150b, and 150c and/or macronutrients 140 may be subsequently added to the liquid 102. Liquid 102 also includes oligopeptides 130 which may bioencapsulate micronutrients 150a, 150b, and 150c to help facilitate delivery of micronutrients 150a, 150b, and 150c to parts of a plant.

Biostimulant compositions may include nutrients such as micronutrients and/or macronutrients, some of which are from the plant-based protein source, and some of which are added to the biostimulant composition to enhance the functions of the biostimulant composition.

Example nutrients include but are not limited to calcium, sulfur, magnesium, carbon, oxygen, hydrogen, iron, manganese, boron, molybdenum, zinc, chlorine, sodium, cobalt, and silicon. Examples of micronutrients include iron, manganese, zinc, copper, boron, silicon, and molybdenum. The concentration of each micronutrient including both added micronutrients and existing micronutrients from the plant-based protein source, in the biostimulant composition may be about 1% to about 15%. Macronutrients include nitrogen, phosphorous, potassium, and calcium. The concentration of each macronutrient including both added macronutrients and existing macronutrients from the plant-based protein source, in the biostimulant composition may be about 1% to about 15%, or about 5%.

In specific examples in this paragraph, nitrogen content is from the raw feedstock; no additional nitrogen is added to form the biostimulant composition. In one example, a biostimulant composition has about 5% boron and about 5% nitrogen. In one example, a biostimulant composition has about 5% manganese and about 2% nitrogen. In one example, a biostimulant composition has about 14% potassium and about 1% nitrogen. In one example, a biostimulant composition has about 6% calcium and about 2% nitrogen. In one example, a biostimulant composition has about 4% zinc, about 4% manganese, and about 2% nitrogen. In one example, a biostimulant composition has about 4% zinc and about 3% nitrogen. In some embodiments, nitrogen in these mixtures is from the raw starting material and is not separately added to the composition.

In various embodiments, the biostimulant composition also includes water. In various embodiments, the amount of water in the biostimulant composition is about 1% to about 99%. In various embodiments, biostimulant compositions having any of the above concentrations of components may be diluted in water, such as about 40% water. Dilution of a biostimulant composition may result in a particular ratio of non-water components to water. In some embodiments, dilution or evaporation is performed to obtain a density of about 1 gr/ml to about 3 gr/ml, or about 1.1 gr/ml or about 1.3 gr/ml. In some embodiments, the biostimulant composition is diluted in water such that concentrations of amino acids present in the biostimulant composition are divided in half.

In various embodiments, the free amino acid component of the biostimulant composition includes about 40 wt % to about 60 wt % lysine by weight of the total weight of free amino acids in the biostimulant composition, and about 1 wt % to about 15 wt % boron. In various embodiments, the free amino acid component of the biostimulant composition includes about 40 wt % to about 60 wt % lysine by weight of the total weight of free amino acids in the biostimulant composition, and about 1 wt % to about 15 wt % manganese. In various embodiments, the free amino acid component of the biostimulant composition includes about 40 wt % to about 60 wt % lysine, and about 1 wt % to about 15 wt % zinc. In various embodiments, the free amino acid component of the biostimulant composition includes about 40 wt % to about 60 wt % lysine by weight of the total weight of free amino acids in the biostimulant composition, and about 1 wt % to about 15 wt % calcium. In various embodiments, the free amino acid component of the biostimulant composition includes about 40 wt % to about 60 wt % lysine by weight of the total weight of free amino acids in the biostimulant composition, and about 1 wt % to about 15 wt % manganese. In various embodiments, the free amino acid component of the biostimulant composition includes about 40 wt % to about 60 wt % lysine by weight of the total weight of free amino acids in the biostimulant composition, about 1 wt % to about 15 wt % manganese, and about 1 wt % to about 15 wt % zinc.

In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine, and about 1 wt % to about 15 wt %. In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine, and about 1 wt % to about 15 wt % manganese. In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine, and about 1 wt % to about 15 wt % zinc. In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine, and about 1 wt % to about 15 wt % calcium. In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine, and about 1 wt % to about 15 wt % manganese. In various embodiments, the free amino acid component of the biostimulant composition includes about 40 wt % to about 60 wt % lysine by weight of the total weight of free amino acids in the biostimulant composition, about 30 wt % to about 40 wt % glutamic acid and glutamine by weight of the total weight of free amino acids in the biostimulant composition, about 1 wt % to about 15 wt % manganese, and about 1 wt % to about 15 wt % zinc.

In one example, a 1 Liter (L) biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 14% added water-soluble potassium including potassium that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble boron including boron that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 6% added water-soluble calcium including calcium that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble manganese including manganese that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble magnesium (e.g., MgO) including magnesium that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 5% added water-soluble zinc including zinc that may have been from the plant-based protein source, and has about 10% free amino acids of the total 1 L of biostimulant.

In one example, a 1 L biostimulant includes 550 ml of water, and 450 ml of biostimulant composition (e.g., amino acids, oligopeptides, phytohormones, micronutrients, macronutrients, and other components derived from the plant-based protein source) before added micronutrients and/or macronutrients, includes about 4% added water-soluble zinc including zinc that may have been from the plant-based protein source, and about 4% added water-soluble manganese including manganese and has about 10% free amino acids of the total 1 L of biostimulant.

Biostimulant compositions described herein may be packaged in liquid form of bottles of various sizes, including but not limited to 1 L bottles, 5 L bottles, 20 L bottles, and 1000 L bottles.

Methods of Making Biostimulant Compositions

Biostimulant compositions described herein are made using any of various methods. In some embodiments, the compositions are made by conducting enzymatic hydrolysis of a plant-based protein source and by adding supplemental micronutrients to the composition, either before or after the hydrolysis. The enzymatic hydrolysis converts plant-based protein to free amino acids and, optionally, oligopeptides.

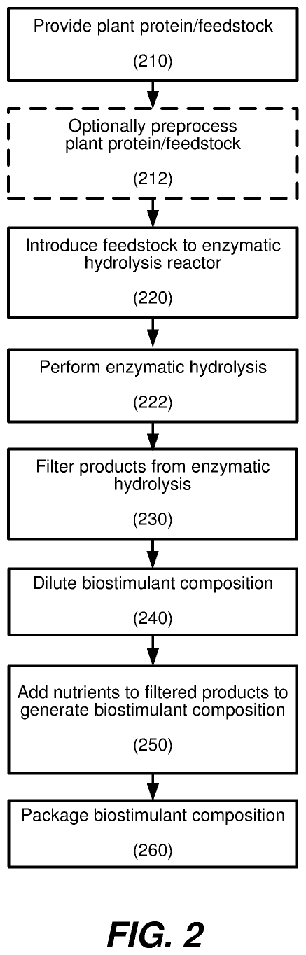

FIG. 2 provides a process flow diagram depicting operations of a method embodiment described herein. In an operation 210, a plant protein and/or feedstock is provided. Example plant sources of feedstock, including plant-based proteins, are described herein and may include but are not limited to carobs, peanuts, rice, soybean, Plukenetia volubilis, and tarwi. The raw plant-based feedstock may be processed (such as ground to a meal), to achieve a feedstock with a particular particle size and water content. In some embodiments, the plant-based feedstock is dried and then milled.

Prior to an operation 220, in an optional operation 212, the pre-processed feedstock (such as a feedstock powder) may undergo pre-hydrolysis processing. Pre-hydrolysis processing may be performed to eliminate polyphenols in vegetable flour because they inhibit the functioning of protease enzymes. Various types of pre-hydrolysis may be performed. Examples include mechanical agitation, addition of water or other liquid, chemical processing such as chemical extraction, sieving, etc. In one example, during pre-hydrolysis processing, polyphenols are extracted from the meal or other feedstock using, e.g., ethanol. Proteases may be mixed with the plant-based feedstock powder. The pH may also be adjusted to make the pH suitable for the enzyme used. In some embodiments, enzymes for conducting enzymatic hydrolysis are added to the feedstock during a preprocessing operation.

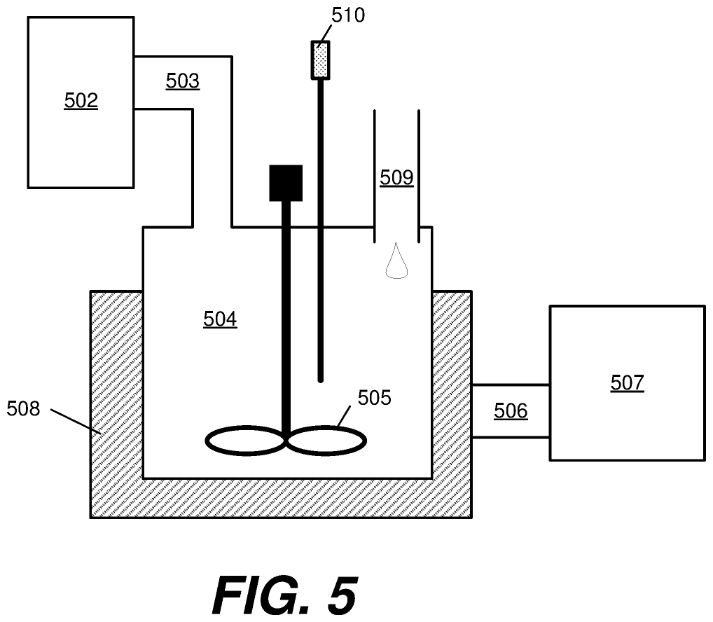

In an operation 220, the feedstock is introduced to an enzymatic hydrolysis reactor. An example is provided in FIG. 5. The enzymatic hydrolysis reactor may include a vessel 504 for containing and/or mixing various components, including processed feedstock and enzymes from a source 502 through inlet 503. In some embodiments, the enzymatic hydrolysis reactor includes a mixing or agitation mechanism such as propeller 505. The reactor also includes pH probe 510 for measuring pH. pH and temperature are controlled in the vessel 504. The pH may be maintained at a pH between 7 and 9, or about 8.5. pH is controlled by including an inlet 509 for dripping acid or base fluids to regulate the pH. For example, 10M of NaOH may be added to maintain a pH of about 8.5. The temperature may be maintained at a temperature between about 55° C. and about 60° C. The temperature may be maintained by using heat sleeve 508. The enzymatic hydrolysis reactor is configured to chemically hydrolyze proteins in the feedstock to produce free amino acids and optionally oligopeptides. Hydrolyzing enzymes are added to the feedstock either before or after the feedstock is introduced to the reactor. Water may be added to the enzymes and/or feedstock either before or after the feedstock is introduced to the reactor. Once, all components are added to the reactor, the temperature and/or pressure of the reactor may be adjusted, and from there, enzymatic hydrolysis proceeds naturally. In some embodiments, the plant-based feedstock includes enzymes, plant-based protein source as a powder, and water.

The type of enzyme used in enzymatic hydrolysis depends on the feedstock and the type of amino acid profile desired for the biostimulant composition. Enzymes are capable of breaking protein chains at a particular hydrolysis reaction rate. One enzyme that may be used is a bioprotease that is a purified liquid enzymatic preparation. Some enzymes are widely available and widely used in the detergent production industry, the food industry, and in the textile industry. Example proteases that may be used for enzymatic hydrolysis include but are not limited to aspartic proteases, serine proteases, thiol proteases, and metalloproteases. Example aspartic proteases include but are not limited to pepsin, pepsin A, chymosin, and renin. Example serine proteases include but are not limited to trypsin, chymotrypsin, subtilisin novo, and alcalase. Example thiol proteases include but are not limited to pure papain and bromelain. Proteases may be derived from one or more of the following sources: ox, pig, calf, papaya, pineapple, Bacillus subtilis, Bacillus lichiniformis, Aspergillus niger, Ananas comosus, and Aspergillus oryzae. Proteases may be provided as a mixture of various types of proteases. For example, a protease that is provided for enzymatic hydrolysis may include a mix of an aspartic protease, a metalloprotease, and a serine protease. Example protease mixtures include but are not limited to ProZyme™ available from PRN Pharmacal in Pensacola, Fla.; Panzyme™ available from Nutra BioGenesis in Park City, Utah Biozyme A™ available from G-Biosciences in St. Louis, Mo., and Sanzyme available from Ciba Giegy of Switzerland.

Returning to FIG. 2, in an operation 222, enzymatic hydrolysis is performed. During enzymatic hydrolysis, the following parameters are monitored and controlled: substrate and enzyme concentration, reaction temperature, pH, and stirring speed. The reference substrate (vegetable flour) concentration of milled feedstock weight to water volume is about 10% to about 15% (p/v). In one example, for enzymatic hydrolysis of carob germ, water is added to 300 grams of carob germ having a dry matter content of 55% to a final volume of 1 L such that the resulting mixtures includes a concentration of protein content of 18% (w/v). The enzyme concentration during enzymatic hydrolysis may be about 0.1% to about 0.2% (v/v) or about 0.15% (v/v). Enzymatic hydrolysis may be performed in the reactor at a temperature of about 45° C. to about 55° C. or up to about 60° C. In some embodiments, the mixture may be mixed for a duration of about 2 hours to about 4 hours. The enzymatic hydrolysis may be performed at standard atmospheric pressure. The pH of the enzymatic hydrolysis is determined by the pH suitable for the protease selected. Some enzymes are suitable for a pH of about 7 to about 11, and some can have maximum activity at a pH of about 9. During enzymatic hydrolysis, concentrated NaOH may be added to maintain the pH in such way so as not to substantially increase the volume in the vessel. Stirring speed may be adjusted throughout the enzymatic hydrolysis process depending on the texture of the hydrolysates. For example, when insoluble material solubilizes, stirring speed may be reduced to accommodate the newly soluble texture of the hydrolysates. Enzymatic hydrolysis may be performed until at least about 10% by weight or at least about 15% by weight or at least about 20% by weight of the amount of proteins in the feedstock is converted to free amino acids, oligopeptides, and peptides.

After the hydrolysis process completes, the hydrolyzed mixture may be optionally centrifuged. The centrifuged hydrolyzed mixture is removed from the reactor which may be performed by delivering via outlet 506 of FIG. 5 to filter 507. While proteinaceous material in the feedstock is broken down by proteases, other material in the feedstock is left wholly or partially unreacted. Examples of such unreacted materials include, micronutrients, macronutrients, phytohormones, and the secondary metabolites.

In some embodiments, after hydrolysis, hydrolyzing enzymes are inactivated by, e.g., a temperature shock. Returning to FIG. 2, in an operation 230, the products from the enzymatic hydrolysis are filtered. In some embodiments, two filtrations are carried out (coarse and fine). The first filtration eliminates solids, and the second eliminates further contaminants and solids which are smaller in size. After filtration, in certain embodiments, the product is concentrated to a density of approximately 1.18 g/ml. Finally, in some embodiments, the resulting product is pasteurized to eliminate microorganism contaminants.

In an operation 240, the biostimulant composition is diluted to an amount such as those described above. In some embodiments, water is added to the biostimulant composition to achieve a water content of at least about 40% by volume.

In an operation 250, nutrients such as micronutrients and/or macronutrients are added to the filtered and diluted products to generate a biostimulant composition. The micronutrients and macronutrients are mixed with the products from the reactor to form a homogeneous mixture, which may prevent particles from sinking to the bottom of the liquid. Mixing may be performed using a paddle or other mechanical component, which may be automatically or manually controlled. Micronutrients include but are not limited to iron, manganese, boron, molybdenum, zinc, chlorine, sodium, and cobalt. One, two, three, or more of the above micronutrients may be added. The amount added may be such that they result in the concentration of each micronutrient including both added micronutrients and existing micronutrients from the plant-based protein source, in the biostimulant composition to be of about 1% to about 15% by weight. Macronutrients include nitrogen, phosphorous, potassium, calcium, sulfur, magnesium, carbon, oxygen, and hydrogen, which may also be added such that the resulting concentration of one or more of the macronutrients is about 1% to about 15% by weight. In some embodiments, macronutrients are not added.

In an operation 260, the diluted biostimulant composition is packaged. As described above, the diluted biostimulant composition may be packaged in liquid form in to containers (e.g., bottles) of any of various sizes, such as 1 L bottles.

Methods of Using Biostimulant Compositions

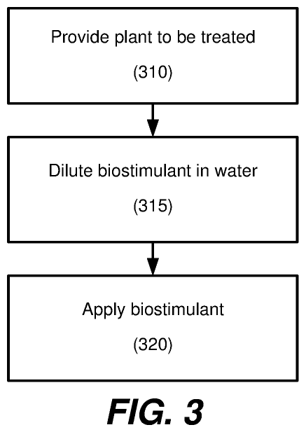

Biostimulant compositions described herein can be applied to crops or plants in various ways. Prior to applying to crops, a biostimulant composition is diluted. FIG. 3 provides a process flow diagram depicting operations that may be performed in accordance with certain embodiments. In operation 310, the plant to be treated is located or provided. The plant can be any one of a variety of crops, both ones having intensive short cycles and extensive long cycles. Examples include but are not limited to vegetables, industrial grains, berries, sugar cane, fruit trees, superfoods, and grapes. Biostimulants are not crop specific and are useful for the vast majority of crops grown, including agricultural, medical and horticultural crops. They can be used in organic or conventional farming. Each plant type can utilize a different application regime of biostimulant, to maximize productivity.

In operation 315, a biostimulant is diluted to an amount such as those described above. In some embodiments, water is added to the biostimulant composition to achieve a water content of at least about 40% by volume.

In operation 320, the diluted biostimulant is applied to a target crop. When the diluted biostimulant is applied depends on the composition of the biostimulant, the amount of diluted biostimulant applied, and the time in the life cycle of the plant that can take advantage of the benefits of the biostimulant composition. Plants undergo various stages of life in their life cycles: seeds, sprouts or germination, seedlings, adult plants that undergo pre-flowering, flowering, pre-fruiting, and/or fruiting. Plants undergo reproduction and pollination, which may involve growth of flowers and/or fruits, prior to seed spreading. Some plants in different parts of their life cycles can use different amounts of a diluted biostimulant. Some plants in different parts of their life cycles can use different amounts of the same biostimulant. Biostimulant compositions can be applied to various parts of a plant, such as the seed, seedling, stem, leaves, branches, flowers, and fruit, and its surroundings, including the soil. The diluted biostimulant may be applied to a plant in a pot, or a plant grown by hydroponics, or a plant grown in an open field. Each of these types of plants may utilize different amounts of biostimulant.

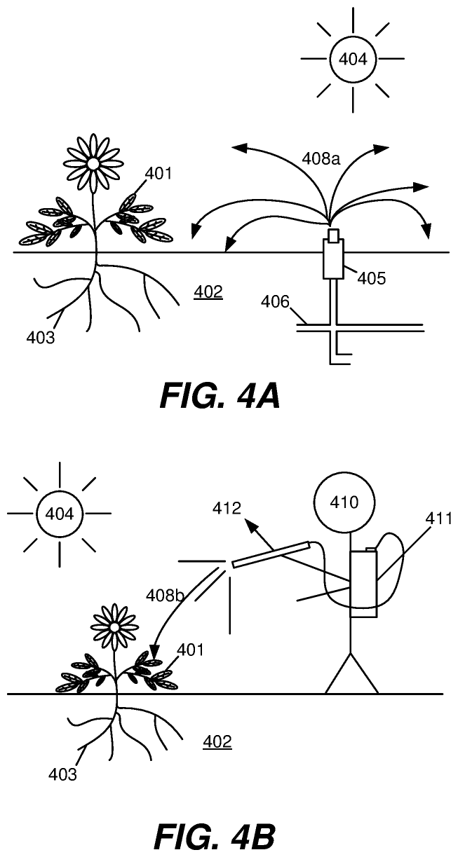

The location in which the diluted biostimulant is applied may also vary from plant to plant. For example, in some embodiments, irrigation systems are used, such as shown in the example in FIG. 4A, which includes a schematic diagram of a plant 401 having roots 403 in soil 402 under a light source 404 (in this case, the sun), with an irrigation system having piping 406 and delivery spout 405 whereby the trajectory 408a of a diluted biostimulant may be used to apply the diluted biostimulant via irrigation.

In some embodiments, diluted biostimulants are applied directly to a plant, such as to the leaves or the foliage of a plant and may be manually applied by a person. An example is provided in FIG. 4B which is a schematic diagram of a plant 401 having roots 403 in soil 402 under a light source 404 whereby the trajectory 408b of a diluted biostimulant is delivered or sprayed via a mister 412 handled by a human 410 from a container 411 of biostimulant. Where the diluted biostimulant is applied depends on environmental variables as well.

### CONCLUSION

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

1. A biostimulant composition for applying to plants, the biostimulant composition comprising:
two or more free amino acids derived from a plant by enzymatic hydrolysis, the two or more free amino acids selected from the group consisting of glutamic acid, glycine, and lysine;
oligopeptides derived from the plant by enzymatic hydrolysis; and
one or more nutrients comprising at least one micronutrient selected from the group consisting of iron, manganese, boron, molybdenum, zinc, chlorine, sodium, and cobalt,
wherein the plant is a non-carob plant.

2. The biostimulant composition of claim 1, wherein the one or more nutrients comprises a macronutrient selected from the group consisting of potassium and calcium.

3. The biostimulant composition of claim 2, further comprising water, wherein the macronutrient has a concentration of at least about 5%.

4. The biostimulant composition of claim 2, further comprising water, wherein the macronutrient is potassium and has a concentration of at least about 10%.

5. The biostimulant composition of claim 1, wherein the at least one micronutrient comprises boron.

6. (canceled)

7. The biostimulant composition of claim 1, wherein the at least one micronutrient comprises manganese.

8. (canceled)

9. The biostimulant composition of claim 1, wherein the one or more nutrients comprises magnesium.

10. (canceled)

11. The biostimulant composition of claim 1, wherein the at least one micronutrient comprises zinc.

12. (canceled)

13. (canceled)

14. (canceled)

15. The biostimulant composition of claim 1, wherein the plant is selected from the group consisting of peanut, tarwi, carob germ, soybean, and Plukenetia volubilis.

16. The biostimulant composition of claim 1, further comprising water.

17. (canceled)

18. The biostimulant composition of claim 1, wherein the two or more free amino acids comprise at least glutamic acid and glutamine, glycine, threonine, alanine, leucine, and lysine.

19. The biostimulant composition of claim 1, wherein the two or more free amino acids comprise trace amounts of one or more secondary amino acid selected from the group consisting of aspartic acid, serine, tyrosine, arginine, valine, tryptophan, phenylalanine, asparagine, and isoleucine.

20. (canceled)

21. The biostimulant composition of claim 1, further comprising one or more phytohormones derived from the plant.

22. (canceled)

23. (canceled)

24. (canceled)

25. (canceled)

26. The biostimulant composition of claim 1, wherein the enzymatic hydrolysis is catalyzed by a protease.

27. (canceled)

28. (canceled)

29. (canceled)

30. (canceled)

31. A biostimulant composition for applying to plants, the biostimulant composition comprising:
free amino acids comprising:
free glutamic acid and glutamine having a weight percent of about 30% to about 40% of the total free amino acid weight in the biostimulant composition,
glycine having a weight percent of about 10% to about 20% of the total free amino acid weight in the biostimulant composition, and
lysine having a weight percent of about 40% to about 60% of the total free amino acid weight in the biostimulant composition;
at least one oligopeptide; and
a nutrient.

32. The biostimulant composition of claim 31, wherein the nutrient is selected from the group consisting of calcium, potassium, sulfur, magnesium, carbon, oxygen, hydrogen, iron, manganese, boron, molybdenum, zinc, chlorine, sodium, and cobalt.

33. The biostimulant composition of claim 31, further comprising a phytohormone.

34. (canceled)

35. A method of producing a biostimulant composition comprising:
receiving a plant-based feedstock including a plant protein;
enzymatically hydrolyzing the plant protein to produce a hydrolysis product comprising an amino acid and an oligopeptide; and
adding one or more nutrients to the plant-based feedstock or hydrolysis product.

36. The method of claim 35, wherein the plant-based feedstock is selected from the group consisting of legumes, tarwi, peanut, and Plukenetia volubilis.

37. The method of claim 35, wherein the one or more nutrients are selected from the group consisting of calcium, potassium, sulfur, magnesium, carbon, oxygen, hydrogen, iron, manganese, boron, molybdenum, zinc, chlorine, sodium, and cobalt.

38. (canceled)

39. (canceled)

40. (canceled)

41. (canceled)
